(19) United States
(12) Patent Application Publication
(10) Pub. No.: US 2015/0051437 A1
(43) Pub. Date: Feb. 19, 2015

(54) VENTRICULAR ASSIST SYSTEM

(71) Applicants: Takayuki Miyakoshi, Nagano (JP); Shinji Kobayashi, Nagano (JP); Hideki Kanebako, Nagano (JP); Tomoya Kitano, Nagano (JP)

(72) Inventors: Takayuki Miyakoshi, Nagano (JP); Shinji Kobayashi, Nagano (JP); Hideki Kanebako, Nagano (JP); Tomoya Kitano, Nagano (JP)

(73) Assignee: SUN MEDICAL TECHNOLOGY RESEARCH CORPORATION, Suwa-shi (JP)

(21) Appl. No.: 14/388,242

(22) PCT Filed: Mar. 27, 2012
(86) PCT No.: PCT/JP2012/058000

§ 371 Date: Sep. 26, 2014

(51) Int. Cl. A61M 1/10; A61M 1/12

(52) U.S. Cl. CPC A61M 1/1008; A61M 1/1012; A61M 1/122
USPC 600/16

(57) ABSTRACT

A ventricular assist system includes: a ventricular assist blood pump provided with a rotational part having an impeller and a housing which houses the rotational part therein; an introduction-side artificial vessel which introduces a liquid into the ventricular assist blood pump; and a delivery-side artificial vessel which delivers the liquid from the ventricular assist system. In the ventricular assist system, a difference between a maximum flow rate and a minimum flow rate of a liquid in a state where the ventricular assist system is connected to a liquid-discharge source which discharges the liquid while increasing and decreasing the flow rate of the liquid at a fixed cycle is 40% or more of a difference between the maximum flow rate and the minimum flow rate of the liquid in a state where the ventricular assist system is not connected to the liquid-discharge source.

### TECHNICAL FIELD

The present invention relates to a ventricular assist system.

### BACKGROUND ART

Conventionally, there have been known a ventricular assist system which includes: a ventricular assist blood pump provided with a rotational part having an impeller and a housing which houses the rotational part therein; an introduction-side artificial vessel which introduces a liquid to the ventricular assist blood pump; and a delivery-side artificial vessel which delivers the liquid from the ventricular assist blood pump (see patent literature 1 and non-patent literature 1, for example)

FIG. 5 is an exploded perspective view of a ventricular assist blood pump 900 in a conventional ventricular assist system. As shown in FIG. 5, the ventricular assist blood pump 900 includes: a rotational part 910 having an impeller 912; and housings 920, 922 which house the rotational part 910 therein. Such a conventional ventricular assist system can assist an action of a heart of a patient having cardiopathy during a period till he receives a heart transplant.

### PRIOR ART LITERATURE

### Patent Literature

Patent literature 1: JP-A-2009-523488
Non-patent literature 1: Jeffrey A LaRose and three others, “American Society of Artificial Internal Organs journal”, 2010, 56, No. 4, p. 285-289

### SUMMARY OF THE INVENTION

### Task to be Solved by the Invention

Cardiopathy is a disease which is very difficult to cure. At present, in many cases, only way to fundamentally cure such cardiopathy is with a heart transplant. However, it is a rare case where conditions necessary for carrying out the heart transplant (for example, the appearance of a donor who is compatible with a patient) are met readily. That is, under current circumstances, a patient waiting for a heart transplant (heart transplant waiting patient) has to wait for a donor who is compatible with the patient for a long period. Accordingly, there may be a case where a period until a heart transplant is carried out is extremely prolonged so that a patient cannot have a heart transplant eternally. In view of such circumstances, there has been proposed an idea that a patient continues the use of a ventricular assist system until he passes away without receiving a heart transplant.

As described above, there is a tendency that a period where a user of a ventricular assist system (hereinafter simply referred to as “user”) uses the ventricular assist system is becoming longer than a period which has been conventionally estimated. Accordingly, the importance of suppressing the degree at which the health of a user deteriorates during a long-term use is steadily increasing.

Accordingly, it is an object of the present invention to provide a ventricular assist system which can suppress the degree at which she health of a user deteriorates during long-term use compared to a conventional ventricular assist system.

### Means for Solving the Task

Inventors of the present invention have come up with an idea that the pulsatility of a blood flow discharged from a ventricular assist system is an important factor to be taken into consideration in suppressing the degree at which the health of a user deteriorates, and the present invention has been made under such finding. That is, a ventricular assist blood pump provided with a rotation part rotates a rotation part at a fixed rotational speed and hence, the ventricular assist blood pump essentially produces a blood flow having no pulsatility. However, a heart moves blood by expansion and contraction (beat) of muscles thereof and hence, from a viewpoint of the health of a user, it is preferable that the blood flow has pulsatility. The present invention relates to a ventricular assist system which can make use of pulsatility of a blood flow generated by heart beat while using a ventricular assist blood pump provided with a rotation part. The ventricular assist system according to the present invention has the following constitution.

(1) A ventricular assist system of the present invention includes: a ventricular assist blood pump provided with a rotational part having an impeller and a housing which houses the rotational part therein; an introduction-side artificial vessel which introduces a liquid into the ventricular assist blood pump; and a delivery-side artificial vessel which delivers the liquid from the ventricular assist system, and is characterized in that a difference between a maximum flow rate and a minimum flow rate of the liquid in a state where the ventricular assist system is connected to a liquid-discharge source which discharges the liquid while increasing and decreasing the flow rate of the liquid at a fixed cycle is 40% or more of a difference between the maximum flow rate and the minimum flow rate of the liquid in a state where the ventricular assist system is not connected to the liquid-discharge source.

According to the ventricular assist system of the present invention, a difference between the maximum flow rate and the minimum flow rate of the liquid in a state where the ventricular assist system is connected to the liquid-discharge source is 40% or more of a difference between the maximum flow rate and the minimum flow rate of the liquid in a state where the ventricular assist system is not connected to the liquid-discharge source and hence, a change in flow rate is sufficiently large with respect to a change in head. As a result, compared to the conventional ventricular assist system, it is possible to suppress the degree at which the health of a user deteriorates during long-term use.

In view of the above, it is preferable that a difference between the maximum flow rate and the minimum flow rate of the liquid discharged from the ventricular assist system is 60% or more of a difference between the maximum flow rate and the minimum flow rate of the liquid in a state where the ventricular assist system is not connected to the liquid-discharge source, and it is more preferable that the percentage is 80% or more. Further, it is needless to say that 100% is the most preferable as an ideal percentage.

“liquid-discharge source” is a heart when a ventricular assist system is actually used in a body and is a device simulating an action of the heart when the ventricular assist system is tested outside the body.

“difference between a maximum flow rate and a minimum flow rate of the liquid in a state where the ventricular assist system is connected” is not calculated based on a flow rate of the liquid obtained when only the ventricular assist blood pump in the ventricular assist system is taken into account (so-called pump flow) but is calculated based on a flow rate obtained when the whole system including the liquid-discharge source, the ventricular assist system and the like is taken into account (so-called total flow).

In this specification, “ventricular assist blood pump” is a main element of the ventricular assist system, and is a pump which assists a heart weakened by a disease by applying a moving force to blood.

“ventricular assist system” is a set of devices which is used in the form that the system is mounted on the heart weakened by a disease, and a system which mainly assists the movement of blood.

“artificial vessel” includes, in its category, both a flexible artificial vessel made of fabric or a soft resin, and a pipe-shaped artificial vessel made of a hard resin or metal.

It is preferable that the ventricular assist system of the present invention is an embedded-type ventricular assist system which is used in an embedded manner in a body in an actual use (that is, the ventricular assist system being so small that the ventricular assist system can be used in an embedded manner in a body).

(2) A ventricular assist system of the present invention includes: a ventricular assist blood pump provided with a rotational part having an impeller and a housing which houses the rotational part therein; an introduction-side artificial vessel which introduces a liquid into the ventricular assist blood pump; and a delivery-side artificial vessel which delivers the liquid from the ventricular assist blood pump, and is characterised in that a relationship between a head and a flow rate is measured using a liquid whose viscosity and density correspond to viscosity and density of blood as a working liquid, and in a graph where the head is taken on an axis of ordinates using mmHg as a unit and the flow rate is taken on an axis of abscissas using L/min as a unit, the flow rate is set to 5 L/min or more at a point where the head is lower than a shutoff head by 20 mmHg in pressure at a fixed rotational speed.

According to the ventricular assist system of the present invention, the flow rate is set to 5 L/min or more at a point where the head is lower than a shutoff head by 20 mmHg in pressure and hence, the flow rate becomes sufficiently large with respect to a magnitude of the head compared to a conventional ventricular assist system whereby the pulsatility of blood flow generated by heart beat can be sufficiently made use of. As a result, compared to the conventional ventricular assist system, the ventricular assist system of the present invention can suppress the degree at which the health of a user deteriorates during long-term use.

In view of the above, it is preferable that the flow rate is set to 8 L/min or more at a point where the head is lower than a shutoff head by 20 mmHg in pressure, and it is more preferable that the flow rate is set to 10 L/min or more at a point where the head is lower than a shutoff head by 20 mmHg in pressure.

The “shutoff head” indicates a head when a flow rate is 0 L/min.

(3) A ventricular assist system of the present invention includes: a ventricular assist blood pump provided with a rotational part having an impeller and a housing which houses the rotational part therein; an introduction-side artificial vessel which introduces a liquid into the ventricular assist blood pump; and a delivery-side artificial vessel which delivers the liquid from the ventricular assist system, and is characterized in that a relationship between a head and a flow rate is measured using a liquid whose viscosity and density correspond to viscosity and density of blood as a working liquid, and in a graph where the head is taken on an axis of ordinates using mmHg as a unit and the flow rate is taken on an axis of abscissas using L/min as a unit, the inclination of the graph is set to a value which falls within a range of −5 to 0 at a point where the head is set to 100 mmHg and the flow rate is set to 5 L/min at a fixed rotational speed.

According to the ventricular assist system of the present invention, under the above-mentioned condition, the inclination of the graph is set to a value which falls within a range of −5 to 0 at a point where the head is set to 100 mmHg and the flow rate is set to 5 L/min. Accordingly, a change in flow rate becomes sufficiently large with respect to a change in head compared to a conventional ventricular assist system and hence, the pulsatility of blood flow generated by heart beat can be sufficiently made use of. As a result, compared to the conventional ventricular assist system, the ventricular assist system of the present invention can suppress the degree at which the health of a user deteriorates during long-term use.

The reason why the inclination of the graph is set to a value which falls within a range of −5 to 0 is as follows. That is, when the inclination of the graph is less than −5, it is difficult to make a change in flow rate sufficiently large with respect to a change in head, while when the inclination of the graph is more than 0, although the head is increased, the flow rate is also increased and hence, the value is not a significant value. In view of the above, it is preferable that the inclination of the graph is set to a value which falls within a range of −4 to 0, and it is more preferable that the inclination of the graph is set to a value which falls within a range of −3 to 0.

(4) A ventricular assist system of the present invention includes: a ventricular assist blood pump provided with a rotational part having an impeller and a. housing which houses the rotational part therein; an introduction-side artificial vessel which introduces a liquid into the ventricular assist blood pump; and a delivery-side artificial vessel which delivers the liquid from the ventricular assist system, and is characterized in that a change in flow rate is large with respect to a change in head when the liquid is made to flow in the ventricular assist system with a rotational speed of the rotational part set to a fixed value.

Due to such a constitution, according to the ventricular assist system of the present invention, since a change in flow rate is large with respect to a change in head (that is, a change in pressure generated by heart beat), the ventricular assist system of the present invention can sufficiently make use of the pulsatility of the blood flow generated by heart beat. Accordingly, compared to the conventional ventricular assist system, the ventricular assist system of the present invention can suppress the degree at which the health of a user deteriorates during long-term use.

“with a rotational speed of the rotational part set to a fixed value” does not means that a rotational speed of the rotational part should be absolutely set to a fixed value but means that the rotational speed is set to a fixed value when there is no change in head.

(5) According to the ventricular assist system of the present invention, a liquid whose viscosity and density correspond to viscosity and density of blood is used as a working liquid, and when a pressure loss is measured in a state where the ventricular assist blood pump is stopped and the flow rate is set to 6 L/min, the pressure loss may preferably be 25 mmHg or less.

Due to such a constitution, the ventricular assist system of the present invention can sufficiently make use of the pulsatility of the blood flow by making a pressure loss sufficiently low.

It is more preferable that the pressure loss of the ventricular assist system falls within a range of 5 mmHg to 20 mmHg. The reason is as follows. When the pressure loss is larger than 20 mmHg, there may be a case where it is difficult to make use of the pulsatility of the blood flow by making the pressure loss sufficiently low. On the other hand, when the pressure loss is less than 5 mmHg, there may be a case where a force to move blood cannot be sufficiently ensured due to a problem on designing the rotational part.

In this specification, “pressure loss of the ventricular assist system” means a pressure necessary for a working liquid to pass through a flow path from the introduction-side artificial vessel to the delivery-side artificial vessel via the ventricular assist blood pump when the working liquid is made to flow at a predetermined flow rate (6 L/min) in a state the ventricular assist blood pump in the ventricular assist system is stopped.

(6) According to the ventricular assist system of the present invention, the ventricular assist blood pump is formed of a centrifugal-type ventricular assist blood pump, and a numerical value obtained by dividing a minimum inner diameter between the introduction-side artificial vessel and a blood introducing portion of the ventricular assist blood pump by a diameter of rotation of the impeller may preferably be set to a value which falls within a range of 0.2 to 0.8.

Due to such a constitution, the ventricular assist system of the present invention can make use of the pulsatility of the blood flow by making the pressure loss sufficiently low, and it is possible to provide a sufficiently compact ventricular assist system.

The reason why the numerical value obtained by dividing the minimum inner diameter between the introduction-side artificial vessel and the blood introducing portion of the ventricular assist blood pump by the diameter of rotation of the impeller is set to a value which falls within a range of 0.2 to 0.8 is as follows. That is when the value is less than 0.2, the minimum inner diameter becomes so small that there may be a case where it is difficult to sufficiently make use of the pulsatility of the blood flow by making the pressure loss sufficiently low. On the other hand, when the value is more than 0.3, it is difficult to provide a sufficiently compact ventricular assist system.

(7) According to the ventricular assist system of the present invention, a numerical value obtained by dividing the minimum inner diameter between the delivery-side artificial vessel and a blood delivering portion of the ventricular assist blood pump by a diameter of rotation of the impeller is set to a value which may preferably fall within a range of 0.2 to 0.8.

Due to such a constitution, the ventricular assist system of the present invention can make use of the pulsatility of the blood flow by making the pressure loss sufficiently low, and it is possible to provide a sufficiently compact ventricular assist system.

The reason why the numerical value obtained by dividing the minimum inner diameter between the delivery-side artificial vessel and the blood delivering portion of the ventricular assist blood pump by the diameter of rotation of the impeller is set to a value which falls within a range of 0.2 to 0.8 is as follows. That is, when the value is less than 0.2, the minimum inner diameter becomes so small that there may be a case where it is difficult to sufficiently make use of the pulsatility of the blood flow by making the pressure loss sufficiently low. On the other hand, when the value is more than 0.8, it is difficult to provide a sufficiently compact ventricular assist system.

### BRIEF DESCRIPTIONS OF THE DRAWINGS

FIG. 1(a) and FIG. 1(b) are views for explaining a ventricular assist system 100 according to an embodiment.

FIG. 2(a), FIG. 2(b) and FIG. 2(c) are views for explaining a ventricular assist blood pump 110 in the ventricular assist system 100 according to the embodiment.

FIG. 3(a) and FIG. 3(b) are graphs for explaining a mode of a blood flow which is measured by using the ventricular assist system 100 and liquid discharge source according to the embodiment.

FIG. 4 is a graph for explaining a relationship between a head and a flow rate of the ventricular assist system 100 according to the embodiment.

FIG. 5 is an exploded perspective view of a ventricular assist blood pump 900 according to a conventional ventricular assist system.

### BEST MODE FOR CARRYING OUT THE INVENTION

Hereinafter, a ventricular assist system of the present invention is explained based on an embodiment shown in drawings.

### Embodiment

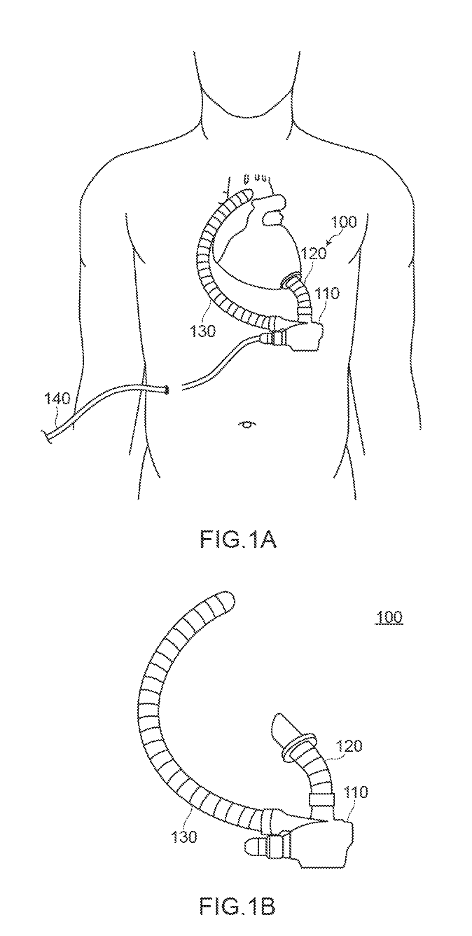

FIG. 1(a) and FIG. 1(b) are views for explaining a ventricular assist system 100 according to an embodiment. FIG. 1(a) is a view showing a mode of the ventricular assist system 100 when the ventricular assist system 100 is actually used, while FIG. 1(b) is a front view showing a ventricular assist pump 110, an introduction-side artificial vessel 120 and a delivery-side artificial vessel 130 taken out from the ventricular assist system 100.

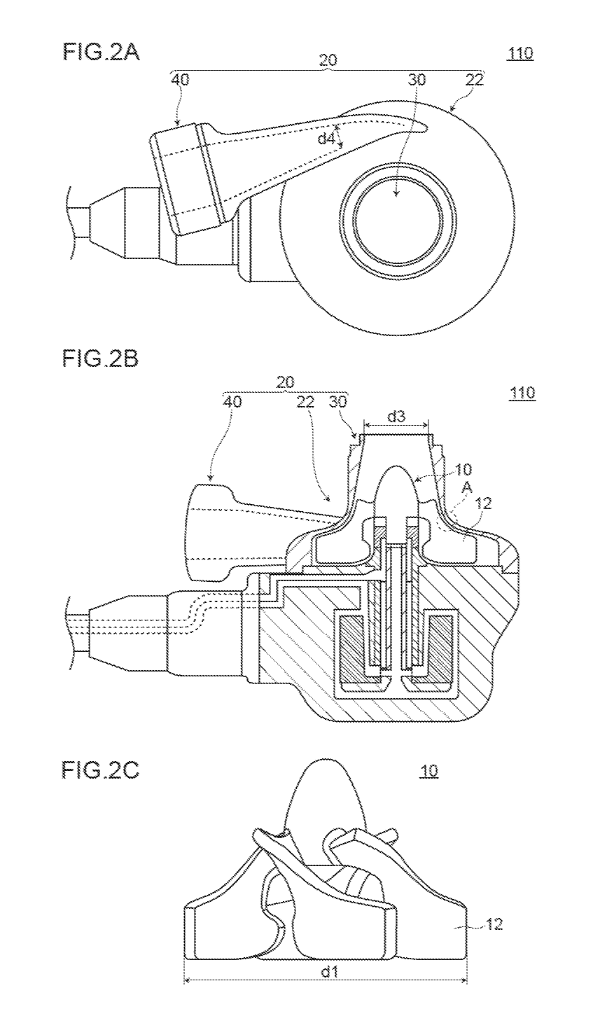

FIG. 2(a), FIG. 2(b) and FIG. 2(c) are views for explaining a ventricular assist blood pump 110 in the ventricular assist system 100 according to the embodiment. FIG. 2(a) is a top plan view of the ventricular assist pump 110, FIG. 2(b) is a cross-sectional view of the ventricular assist pump 110, and FIG. 2(c) is a front view of a rotational part 10.

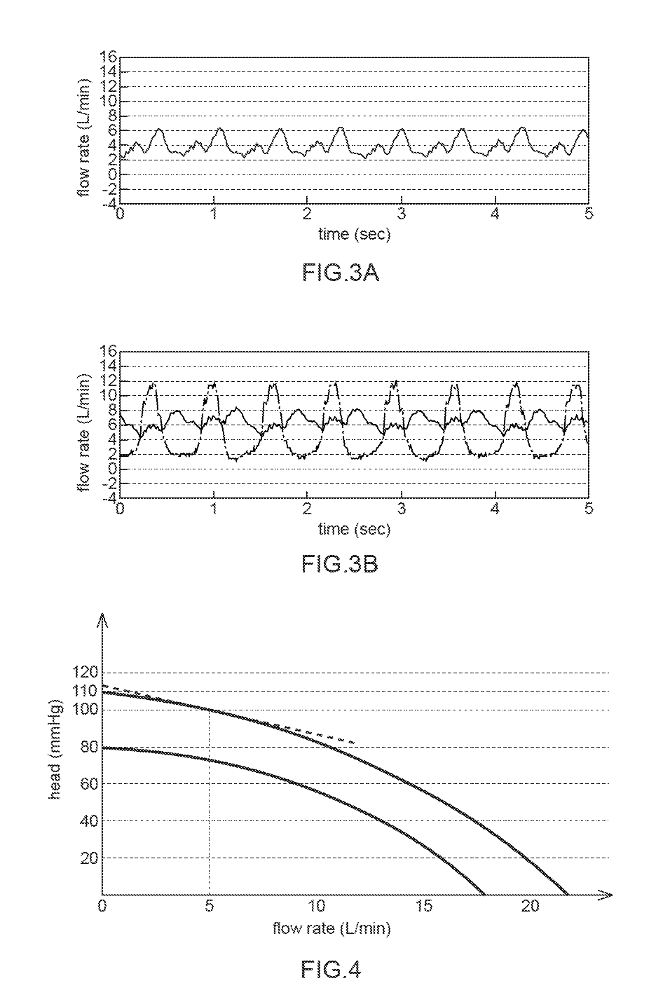

FIG. 3(a) and FIG. 3(b) are graphs for explaining a mode of a blood flow which is measured by using the ventricular assist system 100 and a liquid discharge source according to the embodiment. FIG. 3(a) is a graph showing a mode of blood flow in a state where the ventricular assist system 100 is not connected to a device which simulates a patient's heart suffering a functional disorder (beat simulator), and FIG. 3(b) is a graph showing a mode of the blood flow in a state where the ventricular assist blood pump 110 is connected to the device. In FIG. 3(a) and FIG. 3(b), a flow rate (L/min) is taken on an axis of ordinates, and time (sec) is taken on an axis of abscissas. In the graphs in FIG. 3(a) and FIG. 3(b), a solid line indicates a flow rate of a liquid when the whole system including a liquid discharge source, the ventricular assist system and the like (total flow) is taken into consideration, and a chain line indicates a flow rate when only the ventricular assist blood pump is taken into consideration (pump flow)

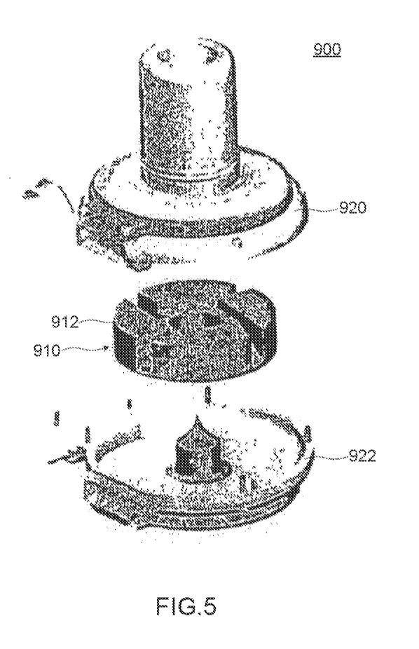

FIG. 4 is a graph for explaining a relationship between a head and a flow rate of the ventricular assist system 100 according to the embodiment. The upper graph is a graph where a flow rate is set to 5 L/min when a head is 100 mmHg, and the lower graph is a graph where a shutoff head is set to 80 mmHg. A broken line which is in contact with the upper graph is a tangent at a point where the head is 100 mHg and the flow rate is 5 L/min.

The ventricular assist system 100 according to the embodiment includes: the ventricular assist blood pump 110; an introduction-side artificial vessel 120; a delivery-side artificial vessel 130; a cable 140; and a control part 150 (not shown in the drawing). The control part 150 is connected to the ventricular assist blood pump 110 by way of the cable 140 and controls the operation of the ventricular assist blood pump 110. The ventricular assist system 100 is an embedded type ventricular assist system which is used in a state were the ventricular assist system 100 is embedded in a human body in an actual use.

As shown in FIG. 2(a), FIG. 2(b) and FIG. 2(c), the ventricular assist blood pump 110 is a centrifugal-type ventricular assist blood pump which includes: a rotational part 10 having an impeller 12 (see FIG. 2(c)); and a housing 20 which houses the rotational part 10 therein. The ventricular assist blood pump 110 further includes, in addition to the constitutional elements described above, a drive part which rotatably drives the rotational part 10, a flow path for a cool sealing liquid (also referred to as a purge liquid, water or saline, for example) which performs functions such as lubrication, cooling and maintaining of a sealing capacity of the inside of the ventricular assist blood pump 110 and the like. However, these constitutional elements are not directly related to the present invention and hence, the explanation of these constitutional elements and the description of symbols in the drawings are omitted.

In the ventricular assist blood pump 110, the rotational part 10 is directly connected to a drive part by way of a rotary shaft. A bearing portion of the rotational part 10 is a mechanical seal and is configured to prevent the intrusion of blood.

The housing 20 includes: a storing part 22 which stores the rotational part; a blood introducing portion 30 which introduces blood into the ventricular assist blood pump 110 from the outside of the ventricular assist blood pump 110; and a blood delivering portion 40 which discharges the blood to the outside of the ventricular assist blood pimp 110 (aorta) from the inside of the ventricular assist blood pump 110. The blood introducing portion 30 is connected to an introduction-side artificial vessel 120, and the blood delivering portion 40 is connected a delivery-side artificial vessel 130. The blood introducing portion and the blood delivering portion may be formed separately from the housing.

The ventricular assist blood pump 110 used in the ventricular assist system 100 according to the embodiment may have following features, for example.

In the ventricular assist blood pump 110, a minimum gap between the impeller 12 and an inner wall of the housing 20 during the operation of the ventricular assist blood pump 110 falls within a range of 0.1 mm to 2.0 mm more preferably falls within a range of 0.5 mm to 0.8 mm. For example, the minimum gap is 0.6 mm.

In the ventricular assist blood pump 110, a liquid whose viscosity and density correspond to viscosity and density of blood is used as a working liquid. When the pressure loss is measured in a state where the ventricular assist blood pump 110 is stopped and the flow rate is set to 6 L/min, the pressure loss is 20 mmHg or less, more preferably falls within a range of 5 mmHg to 16 mmHg. For example, the pressure loss is 14 mmHg.

In the ventricular assist blood pump 110, a numerical value obtained by dividing a volume of the rotational part 10 by a capacity of the housing 20 falls within a range of 0.01 to 0.50, more preferably falls within a range of 0.06 to 0.12. For example, the numerical value is 0.09. “capacity of the housing” does not mean only a capacity of a portion of the housing where the impeller is stored (storing part 22) but means a capacity of the whole housing including a capacity of a portion were blood is introduced into the ventricular assist blood pump 110 (a portion connectable with or separable from the introduction-side artificial vessel) and a capacity of a portion where blood is discharged (a portion connectable with and separable from the delivery-side artificial vessel).

Due to the provision of the ventricular assist blood pump 110 having the above-mentioned constitution, the ventricular assist system 100 can sufficiently make use of the pulsatility of the blood flow by making a pressure loss sufficiently low and hence, the rotational part can sufficiently ensure a force for moving blood.

The introduction-side artificial vessel 120 introduces the liquid to the ventricular assist blood pump 110. In an actual use, the introduction-side artificial vessel 120 connects a heart and the ventricular assist blood pump 110 and introduces blood into the ventricular assist blood pump 110 (see FIG. 1(a)). The introduction-side artificial vessel 120 is a flexible artificial vessel made of fabric or a soft resin and has a length of 7.2 cm, for example.

The delivery-side artificial vessel 130 delivers a liquid from the ventricular assist blood pump 110. In an actual use, the delivery-side, artificial vessel 130 connects the ventricular assist blood pump 110 and an aorta to each other, and delivers blood from the ventricular assist blood pump 110. The delivery-side artificial vessel 130 is a flexible artificial vessel made of fabric or a soft resin and has a length of 25 cm, for example.

In the ventricular assist system 100, when a liquid (blood in actual use in a body) is made to flow in the ventricular assist system 100 with a rotational speed of the rotational part 10 set to a fixed value, a change in flow rate is large with respect to a change in head.

A method of obtaining a graph shown in FIG. 3(a) and a graph shown in FIG. 3(b) is explained. The graphs shown in FIG. 3(a) and FIG. 3(b) are obtained by the following method. That is, a ventricular assist system similar to the ventricular assist system 100 according to the embodiment is actually manufactured, an experiment is performed by connecting the ventricular assist system to a beat simulator which simulates the delivery of blood from a heart (beat simulator), and the result of the experiment is made into graphs. As a working liquid served for the test, a glycerin aqueous solution whose viscosity is prepared to 3.5 cP, for example, is used. The result of graphs (waveforms) reflects disturbance factors such as a pressure spike waveform generated by opening or closing a valve.

As shown in FIG. 3(a), a difference between the maximum flow rate (average maximum flow rate being 6.29 L/min) and the minimum flow rate (average minimum flow rate being 2.45 L/min) of a liquid in a state where the ventricular assist system 100 is not connected to the liquid-discharge source is 3.84 L/min. On the other hand, as shown in FIG. 3(b), a difference between a maximum flow rate (average maximum flow rate being 8.25 L/min) and a minimum flow rate (average minimum flow rate being 4.91 L/min) of a liquid in a state where the ventricular assist system 100 is connected to the liquid-discharge source is 3.34 L/min. Accordingly, in the ventricular assist system 100, the difference between the maximum flow rate and the minimum flow rate of the liquid in a state where the ventricular assist system 100 is connected to the liquid-discharge source which delivers the liquid while increasing and decreasing the flow rate of the liquid at a fixed cycle is 40% or more of the difference between the maximum flow rate and the minimum flow rate of the liquid in a state where the ventricular assist system 100 is not connected to the liquid-discharge source. In the ventricular assist system 100, the percentage is 80% or more. To be specific, the difference between the maximum flow rate and the minimum flow rate of the liquid in a state where the ventricular assist system 100 is connected to the liquid-discharge source which delivers the liquid while increasing and decreasing the flow rate of the liquid at a fixed cycle is approximately 87% of the difference between the maximum flow rate and the minimum flow rate of the liquid in a state where the ventricular assist system 100 is not connected to the liquid-discharge source.

As shown in FIG. 3(b), a difference between a maximum flow rate (average maximum flow rate being 11.73 L/min) and a minimum flow rate (average minimum flow rate being 1.38 L/min) of a pump flow rate in a state where the ventricular assist system 100 is connected to liquid-discharge source is 10.35 L/min. Accordingly, in the ventricular assist system 100, the difference between the maximum flow rate and the minimum flow rate of the pump flow rate in a state where the ventricular assist system 100 is connected to the liquid-discharge source which discharges the liquid while increasing and decreasing the flow rate of the liquid at a fixed cycle is 200% or more of a difference between the maximum flow rate and the minimum flow rate of the liquid in a state where the ventricular assist system 100 is not connected to the liquid-discharge source. In the ventricular assist system 100, the percentage is 250% or more. To be specific, the difference between the maximum flow rate and the minimum flow rate of the pump flow rate in a state where the ventricular assist system 100 is connected to liquid-discharge source is approximately 270% of the difference between the maximum flow rate and the minimum flow rate of the liquid in a state where the ventricular assist system 100 is not connected to the liquid-discharge source.

As described above, the ventricular assist system 100 includes the ventricular assist blood pump 110 where the difference between the maximum flow rate and the minimum flow rate of the pump flow rate in a state where the ventricular assist blood pump is connected to the liquid-discharge source which discharges the liquid while increasing and decreasing the flow rate of the liquid at a fixed cycle is 200% or more of the difference between the maximum flow rate and the minimum flow rate of the liquid in a state where the ventricular assist blood pump is not connected to the liquid-discharge source. Accordingly, a change in flow rate becomes sufficiently large with respect to a change in head. As a result, compared to the conventional ventricular assist system, the ventricular assist system 100 can suppress the degree at which the health of a user deteriorates during long-term use.

A method of obtaining the graph in FIG. 4 is explained. The graph in FIG. 4 is obtained by the following method. That is, a ventricular assist system similar to the ventricular assist system 100 according to the embodiment is manufactured, an experiment is performed using the ventricular assist system, and the result of the experiment is made into a graph. As a working liquid served for the test, a glycerin aqueous solution whose viscosity of is set to 3.5 cP is used.

In the ventricular assist system 100, as shown in FIG. 4, a relationship between a head and a flow rate is measured using a liquid whose viscosity and density correspond to viscosity and density of blood as a working liquid, and when a graph is prepared by taking the head on an axis of ordinates using mmHg as a unit and the flow rate on an axis of abscissas using L/min as a unit at a fixed rotational speed, the flow rate is set to 5 L/min or more at a point where the head is lower than a shutoff head by 20 mmHg, and more particularly is set to 10 L/min or more at such a point.

Further, in the ventricular assist system 100, similarly as shown in FIG. 4, a relationship between a head and a flow rate is measured using a liquid whose viscosity and density correspond to viscosity and density of blood which constitutes a working liquid, and when a graph is prepared by taking the head on an axis of ordinates using mmHg as a unit and the flow rate on an axis of abscissas using L/min as a unit at a fixed rotational speed, the inclination of the graph at a point where the head is 100 mHg and the flow rate is 5 L/min falls within a range of −5 to 0, more particularly −3 to 0. To be specific, the inclination of the graph is approximately −2.6.

In the ventricular assist system 100, by using a liquid whose viscosity and density correspond to viscosity and density or blood as a working liquid, when a pressure loss is measured in a state where the ventricular assist blood pump 110 is stopped and the flow rate is set to 6 L/min, the pressure loss is 25 mmHg or less. In the ventricular assist system 100, the pressure loss falls within a range of 5 mmHg to 20 mmHg. For example, the pressure loss is 18 mmHg.

A diameter of rotation of the impeller 12 (see d1 in FIG. 2(c)) is 40 mm, and a minimum inner diameter between the introduction-side artificial vessel 120 and the blood introducing portion 30 of the ventricular assist blood pump 110 is 16 mm. Accordingly, a numerical value obtained by dividing the minimum inner diameter between the introduction-side artificial vessel 120 and the blood introducing portion 30 of the ventricular assist blood pump ventricular assist blood pump 110 by the diameter of rotation of the impeller 12 falls within a range of 0.2 to 0.8, and to be specific, the numerical value is 0.4. The inner diameter from the introduction-side artificial vessel 120 to the blood introducing portion 30 of the ventricular assist blood pump 110 is uniformly set to 16 mm although the explanation of the inner diameter in conjunction with drawings is omitted, (see also d3 in FIG. 2(b)).

Further, a minimum inner diameter from the delivery-side artificial vessel 130 to the blood delivering portion 40 of the ventricular assist blood pump 110 is 10 mm. Accordingly, in the ventricular assist system 100, a numerical value obtained by dividing the minimum inner diameter from the delivery-side artificial vessel 130 to the blood delivering portion 40 of the ventricular assist blood pump 110 by a diameter of rotation of the impeller 12 falls within a range of 0.2 to 0.8, and to be specific, the numerical value is 0.25. The inner diameter from a distal end portion of the delivery-side artificial vessel 130 to the blood delivering portion 40 of the ventricular assist blood pump 110 is uniformly set to 16 mm although the explanation of the inner diameter in conjunction with drawings is omitted. The inner diameter from the delivery-side artificial vessel 130 to the blood delivering portion 40 of the ventricular assist blood pump 110 becomes minimum in the vicinity of a joint portion between the blood delivering portion 40 and the storing part 22 (back side of the blood delivering portion 40, see d4 in FIG. 2(a)). The minimum inner diameter is a diameter of such a portion.

Hereinafter, advantageous effects of the ventricular assist system 100 according to the embodiment are explained.

According to the ventricular assist system 100 of the embodiment, a difference between the maximum flow rate and the minimum flow rate of the liquid in a state where the ventricular assist system 100 is connected to the liquid-discharge source is 40% or more of a difference between the maximum flow rate and the minimum flow rate of the liquid in a state where the ventricular assist system 100 is not connected to the liquid-discharge source and hence, a change in flow rate is sufficiently large with respect to a change in head. As a result, compared to the conventional ventricular assist system, it is possible to suppress the degree at which the health of a user deteriorates during long-term use.

According to the ventricular assist system 100 of the embodiment, the flow rate is set to 5 L/min or more at a point where the head is lower than a shutoff head by 20 mmHg in pressure and hence, the flow rate becomes sufficiently large with respect to a magnitude of the head compared to a conventional ventricular assist system whereby the pulsatility of blood flow generated by heart beat can be sufficiently made use of. As a result, compared to the conventional ventricular assist system, the ventricular assist system 100 of the present invention can suppress the degree at which the health of a user deteriorates during long-term use.

According to the ventricular assist system 100 of the embodiment, the head is 100 mmHg and the inclination of the graph at a point where the flow rate is 5 L/min falls within a range of −5 to 0 and hence, a change in flow rate becomes sufficiently large with respect to a change in head compared to the conventional ventricular assist system, and the pulsatility of blood flow generated by heart beat can be sufficiently made use of. As a result, compared to the conventional ventricular assist system, it is possible to suppress the degree at which the health of a user deteriorates during long-term use.

According to the ventricular assist system 100 of the embodiment, since a change in flow rate is large with respect to a change in head, the ventricular assist system of the present invention can sufficiently make use of the pulsatility of the blood flow generated by heart beat. Accordingly, compared to the conventional ventricular assist system, the ventricular assist system 100 of the present invention can suppress the degree at which the health of a user deteriorates during long-term use.

According to the ventricular assist system 100 of the embodiment, a liquid whose viscosity and density correspond to viscosity and density of blood is used as a working liquid, and when a pressure loss is measured in a state where the ventricular assist blood pump 110 is stopped and the flow rate is set to 5 L/min, the pressure loss is 25 mmHg or less. Accordingly, it is possible to make the pressure loss sufficiently low and to sufficiently make use of the pulsatility of the blood flow.

According to the ventricular assist system 100 of the embodiment, the ventricular assist blood pump 110 is formed of a centrifugal-type ventricular assist blood pump, and a numerical value obtained by dividing a minimum inner diameter between the introduction-side artificial vessel 120 and a blood introducing portion 30 of the ventricular assist blood pump 110 by a diameter of rotation of the impeller 12 is set to a value which falls within a range of 0.2 to 0.8. Accordingly, it is possible to make use of the pulsatility of the blood flow by making the pressure loss sufficiently low, and it is possible to provide a sufficiently compact ventricular assist system.

According to the ventricular assist system 100 of the embodiment, the ventricular assist blood pump 110 is formed of a centrifugal-type ventricular assist blood pump, and a numerical value obtained by dividing the minimum inner diameter between the delivery-side artificial vessel 130 and a blood delivering portion 40 of the ventricular assist blood pump 110 by a diameter of rotation of the impeller 12 is set to a value which falls within a range of 0.2 to 0.8. Accordingly, it is possible to make the pressure loss sufficiently low and to sufficiently make use of the pulsatility of the blood flow, and it is possible to make a sufficiently compact ventricular assist system.

Although the present invention have been explained in conjunction with the above-described embodiment heretofore, the present invention is not limited to the above-mentioned embodiments, and the present invention can be carried out in various modes without departing from the gist of the present invention. For example, the following modifications can be considered.

(1) The sizes, the numbers, the materials and the shapes of the respective constitutional elements described in the above-mentioned embodiment are merely provided as examples, and can be changed without impairing the advantageous effects of the present invention.

(2) The ventricular assist system 100 of the above-mentioned embodiment has the following four characteristics.

A difference between a maximum flow rate and a minimum flow rate of a liquid in a state where the ventricular assist system is connected to the liquid-discharge source which delivers the liquid while increasing and decreasing the flow rate of the liquid at a fixed cycle is 40% or more of a difference between a maximum flow rate and a minimum flow rate of a liquid in a state where the ventricular assist system is not connected to the liquid-discharge source.

A relationship between a head and a flow rate using a liquid whose viscosity and density correspond to viscosity and density of blood as a working liquid is measured, and when a graph is prepared by taking a head on an axis of ordinates using mmHg as a unit and a flow rate on an axis of abscissas using L/min as a unit at a fixed rotational speed, the flow rate is 5 L/min or more at a point where the head is lower than a shutoff head by 20 mmHg.

A relationship between a head and a flow rate is measured using a liquid whose viscosity and density correspond to viscosity and density of blood as a working liquid, and when a graph is prepared by taking a head on an axis of ordinates using mmHg as a unit and a flow rate on an axis of abscissas using L/min as a unit at a fixed rotational speed, the inclination of the graph at a point where the head is 100 mHg and the flow rate is 5 L/min falls within a range of −5 to 0.

When a liquid is made to flow with a rotational speed of the rotational part 10 set to a fixed value, a change in flow rate is large with respect to a change in head.

However, the present invention is not limited to a ventricular assist system having these characteristics. Provided that a ventricular assist system includes: a ventricular assist blood pimp provided with a rotational part having an impeller and a housing which houses the rotational part therein; an introduction-side artificial vessel which introduces a liquid to the ventricular assist blood pump; and a delivery-side artificial vessel which delivers the liquid from the ventricular assist blood pump, and such a ventricular assist system has any one of the above-mentioned four characteristics, the ventricular assist system falls within the scope of the present invention.

(3) In the above-mentioned embodiment, as an introduction-side artificial vessel and a delivery-side artificial vessel, a flexible artificial vessel made of fabric or a soft resin is used. However, the present invention is not limited to such an artificial vessel. For example, as an introduction-side artificial vessel and a delivery-side artificial vessel, a pipe-like artificial vessel made of a hard resin or metal can be used.

### REFERENCE SIGNS LIST

10: rotational part, 12: impeller, 20: housing, 22: storing part, 30: blood introducing portion, 40: blood delivering portion, 100: ventricular assist system, 110: ventricular assist blood pump, 120: introduction-side artificial vessel, 130: delivery-side artificial vessel, 140: cable

1. A ventricular assist system comprising:
a ventricular assist blood pump provided with a rotational part having an impeller and a housing which houses the rotational part therein;
an introduction-side artificial vessel which introduces a liquid into the ventricular assist blood pump; and
a delivery-side artificial vessel which delivers the liquid from the ventricular assist system, wherein
a difference between a maximum flow rate and a minimum flow rate of the liquid in a state where the ventricular assist system is connected to a liquid-discharge source which discharges while increasing and decreasing the flow rate of the liquid at a fixed cycle is 40% or more of a difference between the maximum flow rate and the minimum flow rate of the liquid in a state where the ventricular assist system is not connected to the liquid-discharge source.

2. A ventricular assist system comprising:
a ventricular assist blood pump provided with a rotational part having an impeller and a housing which houses the rotational part therein;
an introduction-side artificial vessel which introduces a liquid into the ventricular assist blood pump; and
a delivery-side artificial vessel which delivers the liquid from the ventricular assist blood pump, wherein
a relationship between a head and a flow rate is measured using a liquid whose viscosity and density correspond to viscosity and density of blood as a working liquid, and in a graph where the head is taken on an axis of ordinates using mmHg as a unit and the flow rate is taken on an axis of abscissas using L/min as a unit, the flow rate is set to 5 L/min or more at a point where the head is lower than a shutoff head by 20 mmHg in pressure at a fixed rotational speed.

3. A ventricular assist system comprising:
a ventricular assist blood pump provided with a rotational part having an impeller and a housing which houses the rotational part therein;
an introduction-side artificial vessel which introduces a liquid into the ventricular assist blood pump; and
a delivery-side artificial vessel which delivers the liquid from the ventricular assist system, wherein
a relationship between a head and a flow rate is measured using a liquid whose viscosity and density correspond to viscosity and density of blood as a working liquid, and in a graph where the head is taken on an axis of ordinates using mmHg as a unit and the flow rate is taken on an axis of abscissas using L/min as a unit, the inclination of the graph is set to a value which falls within a range of −5 to 0 at a point where the head is set to 100 mmHg and the flow rate is set to 5 L/min at a fixed rotational speed.

4. A ventricular assist system comprising:
a ventricular assist blood pump provided with a rotational part having an impeller and a housing which houses the rotational part therein;
an introduction-side artificial vessel which introduces a liquid into the ventricular assist blood pump; and
a delivery-side artificial vessel which delivers the liquid from the ventricular assist system, wherein
a change in flow rate is large with respect to a change in head when the liquid is made to flow in the ventricular assist system with a rotational speed of the rotational part set to a fixed value.

5. The ventricular assist system according to claim 1, wherein a liquid whose viscosity and density correspond to viscosity and density of blood is used as a working liquid, and when a pressure loss is measured in a state where the ventricular assist blood pump is stopped and the flow rate is set to 6 L/min, the pressure loss is 25 mmHg or less.

6. The ventricular assist system according to claim 1, wherein the ventricular assist blood pump is formed of a centrifugal-type ventricular assist blood pump, and a numerical value obtained by dividing a minimum inner diameter between the introduction-side artificial vessel and a blood introducing portion of the ventricular assist blood pump by a diameter of rotation of the impeller is set to a value which falls within a range of 0.2 to 0.8.

7. The ventricular assist system according to claim 1, wherein the ventricular assist blood pump is formed of a centrifugal-type ventricular assist blood pump, and a numerical value obtained by dividing the minimum inner diameter between the delivery-side artificial vessel and a blood delivering portion of the ventricular assist blood pump by a diameter of rotation of the impeller is set to a value which falls within a range of 0.2 to 0.8.
